(19) United States
(12) Patent Application Publication
(10) Pub. No.: US 2003/0213012 A1
(43) Pub. Date: Nov. 13, 2003

(54) Pesticidal agents

(76) Inventors: Paul Jarrett, Warwickshire (GB); Deborah June Ellis, Warwickshire (GB); James Alun Wynne Morgan, Swansea (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/365,319

(22) Filed: Feb. 12, 2003

Related U.S. Application Data
(62) Division of application No. 09/242,843, filed on Nov. 18, 1999.
Application No. 09/242,843 is a 371 of international application No. PCT/GB97/02284, filed on Aug. 27, 1997.

(30) Foreign Application Priority Data
Aug. 29, 1996 (GB) 9618083.1

(51) Int. Cl. A01H 1/00; C12N 7/00; A01N 65/00; C12N 15/74; C07K 14/195; C12N 1/21; C12P 21/02; C12N 5/04

(52) U.S. Cl. 800/279; 424/94.4; 530/350; 435/69.1; 435/320.1; 435/419; 435/235.1; 435/252.3; 514/2

(57) ABSTRACT

A method for killing pests (e.g. insects) comprising administering material from Xenorhabdus species (e.g. X. nematophilus) such as cells or supernatants orally to the pests, either alone or in conjunction with Bacillus thuringiensis or pesticidal materials derived therefrom. Also disclosed is an isolated pesticidal agent (and compositions comprising the same) characterized in that it is obtainable from cultures of X. nematophilus or mutants thereof, has oral pesticidal activity agent Pieris brassicae, Pieris rapae and Plutella xylostella, is substantially heat stable to 55° C., is proteinaceous, acts synergistically with B. thuringiensis cells as an oral pesticide and is substantially resistant to proteolysis by trypsin and proteinase K. DNA encoding pesticidal activity is also disclosed.

The present invention relates to materials, agents and compositions having pesticidal activity which derive from bacteria, and more particularly from Xenorhabdus species. The invention further relates to organisms and methods employing such compounds and compositions.

There is an ongoing requirement for materials, agents, compositions and organisms having pesticidal activity, for instance for use in crop protection or insect-mediated disease control. Novel materials are required to overcome the problem of resistence to existing pesticides. Ideally such materials are cheap to produce, stable, have a high toxicity (either when used alone or in combination) and are effective when taken orally by the pest target. Thus any invention which provided materials, agents, compositions or organisms in which any of these properties was enhanced would represent a step forward in the art.

Xenorhabdus spp. in nature are frequently symbiotically associated with a nematode host, and it is known that this association may be used to control pest activity. For instance, it is known that certain Xenorhabdus spp. alone are capable of killing an insect host when injected into the host's hemocoel.

In addition, one extracellular insecticidal toxin from Photorhabdus luminescens has been isolated (this species was recently removed from the genus Xenorhabdus, and is closely related to the species therein). This toxin is not effective when ingested, but is highly toxic when injected into certain insect larvae (see Parasites and Pathogens of Insects Vol.2, Eds. Beckage, N. E. et al., Academic Press 1993).

Also known are certain low-molecular weight heterocyclic compounds from P. luminescens and X. nematophilus which have antibiotic properties when applied intravenously or topically (see Rhodes, S. H. et al., PCT WO 84/01775).

Unfortunately none of these prior art materials have the ideal pesticide characteristics discussed above, and in particular, they do not have toxic activity when administered orally.

The present invention provides pesticidal agents and compositions from Xenorhabdus species, organisms which produce such compounds and compositions, and methods which employ these agents, compositions and organisms, that alleviate some of the problems with the prior art.

According to one aspect of the present invention there is disclosed a method of killing or controlling insect pests comprising administering cells from Xenorhabdus species or pesticidal materials derived or obtainable therefrom, orally to the pests.

A PCT application of CSIRO published as WO 95/00647 discloses an apparently toxic protein from Xenorhabdus nematophilus; however no details of the protein's toxicity are given, and certainly there is no disclosure of its use as an oral insecticide.

Thus the invention provides an insecticidal composition adapted for oral administration to an insect, which composition comprises a pesticidal material obtainable from a Xenorhabdus species, or a pesticidal fragment thereof, or a pesticidal variant or derivative of either of these.

The composition may in fact comprise cells of Xenorhabdus or alternatively supernatant taken from cultures of cells of Xenorhabdus species. However, the composition preferably comprises toxins isolable from Xenorhabdus as illustrated hereinafter. Toxic activity has been associated with material encoded by the nucleotide sequence of FIG. 2. Thus, the composition suitably comprises a pesticidal material which is encoded by all or part of the nucleotide sequence of FIG. 2. Pesticidal fragments as well as variants or derivatives of such toxins may also be employed.

The sequence of FIG. 2 is of the order of 40 kb in length. It is believed that this sequence may encode more than one protein, each of which may regulate or be insecticidal either alone or when presented together. It is a matter of routine to determine which parts are necessary or sufficient for insecticidal activity.

As used herein the term “variant” refers to toxins which have modified amino acid sequence but which share similar activity. Certain amino acids may be replaced with different amino acids without altering the nature of the activity in a significant way. The replacement may be by way of “conservative substitution” where an amino acid is replaced with an amino acid of broadly similar properties, or there may be some non-conservative substitutions. In general however, the variants will be at least 60% homologous to the native toxin, suitably at least 70% homologous and more preferably at least 90% homologous.

The term “derivative” relates to toxins which have been modified for example by chemical or biological methods.

These toxins are novel, and they and the nucleic acids which encode them form a further aspect of the invention.

A preferred Xenorhabdus species is the bacteria X. nematophilus. Particular strains of X. nematophilus which are useful in the context of the invention are ATTC 19061 strain, available from the National Collection of Industrial and Marine Bacteria, Aberdeen, Scotland (NCIMB). In addition, suitable strains include two novel strains of Xenorhabdus which were deposited at the NCIMB on 10 Jul. 1997 and were designated with repository numbers NCIMB 40886 and NCIMB 40887. These latter strains form a further aspect of the invention.

All strains have common characteristics as set out in the following Table 1.

TABLE 1

| | Strains | | |
|---|---|---|---|
| Characteristics | ATCC 19061 | NCIMB 40887 | NCIMB 40886 |
| Grain strain | negative | negative | negative |
| Shape/size | rods up to | rods up to | rods up to |
| | 4 μm long | 4 μm long | 4 μm long |
| Motile | Yes | Yes | Yes |
| Bioluminescent | No | No | No |
| Colour on NBTA* | blue | blue | blue |
| insecticidal on | yes | yes | yes |
| ingestion by | | | |
| insects | | | |
| Production of | yes | yes | yes |
| Antibiotics | | | |
| Resistant to | yes | yes | yes |
| ampicillin | | | |
| (50 μg/ml) | | | |
| colony | circular | circular | circular |
| morphology/ | convex | convex | convex |
| colour | cream | cream | cream |

*NBTA (Oxoid nutrient agar containing 0.0025% bromothymol blue and 0.004% tetrazolium chloride)

Preferably the pest target is an insect, and more preferably it is of the order Lepidoptera, particularly Pieris brassicae, Pieris rapae, or Plutella xylostella or the order Diptera, particularly Culex quinquefaciatus.

In a preferred embodiment of the invention, cells from Xenorhabdus species or agents derived therefrom are used in conjunction with Bacillus thuringiensis as an oral pesticide.

In further embodiments, rather than using Bacillus thuringiensis itself, pesticidal materials obtainable from B. thuringiensis (e.g. delta endotoxins or other isolates) are used in conjunction with Xenorhabdus species.

The term ‘obtainable from’ is intended to embrace not only materials which have been isolated directly from the bacterium in question, but also those which have been subsequently cloned into and produced by other organisms.

Thus the unexpected discovery that bacteria of the genus Xenorhabdus (and materials derived therefrom) have pesticidal activity when ingested, and that such bacteria and materials can be used advantageously in conjunction with B. thuringiensis (and toxins or materials derived therefrom), forms the basis of a further aspect of the present invention. The pesticidal activity of B. thuringiensis isolates alone have been well documented. However, synergistic pesticidal activity between such isolates and bacteria of the Xenorhabdus species (or materials derived therefrom) has not previously been demonstrated.

In still further embodiments of the invention, culture supernatant taken from cultures of Xenorhabdus species, particularly X. nematophilus, is used in place of cells from Xenorhabdus species in the methods above.

All of these methods can be employed, inter alia, in pest control.

The invention also makes available pesticidal compositions comprising cells from Xenorhabdus species, preferably X. nematophilus, in combination with B. thuringiensis. As with the methods above, a pesticidal toxin from B. thuringiensis (preferably a delta endotoxin) may be used as an alternative to B. thuringiensis in the compositions of the present invention

Likewise, culture supernatant taken from cultures of Xenorhabdus species, preferably, X. nematophilus may be used in place of cells from Xenorhabdus species.

Such compositions can be employed, inter alia, for crop protection eg. by spraying crops, or for livestock protection. In addition, compositions of the invention may be used in vector control.

The invention further encompasses novel pesticidal agents which can be isolated from Xenorhabdus spp. Techniques for isolating such agents would be understood by the skilled person.

In particular, such techniques include the separation and identification of toxin proteins either at the protein level or at the DNA level.

The applicants have cloned and partially sequenced a region of DNA from Xenorhabdus NCIMB 40887 which region codes for insecticidal activity and this is shown as FIG. 2 (SEQ ID NO. 1) hereinafter. Thus in a preferred embodiment the invention also provides a toxin which is encoded by DNA of SEQ ID No. 1 or a variant or fragment thereof.

The invention also provides a recombinant DNA which encodes such a toxin. The recombinant DNA of the invention may comprise the sequence of FIG. 2 or a variant or fragment thereof. Other DNA sequences may encode similar proteins as a result of the degeneracy of the genetic code. All such sequences are encompassed by the invention.

The sequence provided herein is sufficient to allow probes to be produced which can be used to identify and subsequently to extract DNA of toxin genes. This DNA may then be cloned into vectors and host cells as is understood in the art.

DNA which comprises or hybridises with the sequence of FIG. 2 under stringent conditions forms a further aspect of the invention.

The expression “hybridises with” means that the nucleotide sequence will anneal to all or part of the sequence of FIG. 2 under stringent hybridisation conditions, for example those illustrated in “Molecular Cloning”, A Laboratory Manual” by Sambrook, Fritsch and Maniatis, Cold Spring Habor Laboratory Press, Cold Spring Harbor, N.Y.

The length of the sequence used in any particular analytical technique will depend upon the nature of the technique, the degree of complementarity of the sequence, the nature of the sequence and particularly the GC content of the probe or primer and the particular hybridisation conditions employed. Under high stringency, only sequences which are completely complementary will bind but under low stringency conditions, sequences which are 60% homologous to the target sequence, more suitably 80% homologous, will bind. Both high and low stringency conditions are encompassed by the term “stringent conditions” used herein.

Suitable fragments of the DNA of FIG. 2, i.e. those which encode pesticidal agents may be identified using standard techniques. For example, transposon mutagenesis techniques may be used, for example as described by H. S. Siefert et al., Proc. Natl. Acad. Sci. USA, (1986) 83, 735-739. Vectors such as the cosmid cHRIMI, can be mutated using a variety of transposons and then screened for loss of insectidal activity. In this way regions of DNA encoding proteins responsible for toxic activity can be identified.

For example, the mini-transposon mTn3(HIS3) can be introduced into a toxic Xenorhabdus clone such as cHRIM1, hereinafter referred to as ‘clone 1’, by electroporating cHRIM1 DNA into E.coli RDP146(pLB101) and mating this strain with E.coli RDP146(pOX38), followed by E. coli NS2114Sm. The final strain will contain cHRIM1DNA with a single insertion of the transposon mTn3(HIS3). These colonies can be cultured and tested for insecticidal activity as described in Example 8 hereinafter. Restriction mapping or DNA sequencing can be used to identify the insertion point of mTn3(HIS3) and hence the regions of DNA involved in toxicity. Similar approached can be used with other transposons such as Tn5 and mTn5.

Site directed mutagenesis of cHRIM1 as outlined in “Molecular Cloning, A Laboratory Manual” by Maniatis, Fritsch and Sambrook, (1982) Cold Spring Harbor, can also be used to test the importance of specific regions of DNA for toxic activity.

Alternatively, subcloning techniques can be used to identify regions of the cloned DNA which code for insecticidal activity. In this method, specific smaller fragments of the DNA are subcloned and the activity determined. To do this, cosmid DNA can be cut with a suitable restriction enzyme and ligated into a compatible restriction site on a plasmid vector, such as pUC19. The ligation mix can be transformed into E. coli and transformed clones selected using a selection marker such as antibiotic resistance, which is coded for on the plasmid vector. Details of these techniques are described for example in Maniatis et al, supra, (see p390-391) and Methods in Molecular Biology, by L. G. Davies, M. D. Dibner and J. F. Battey, Elsevier, (see p222-224).

Individual colonies containing specific cloned fragments can be cultured and tested for activity as described in Example 8 hereinafter. Subclones with insecticidal activity can be further truncated using the same methodology to further identify regions of the DNA coding for activity.

The invention also discloses an isolated pesticidal agent characterised in that the agent is obtainable from cultures of X. nematophilus or variants thereof, has oral pesticidal activity against Pieris brassicae, Pieris rapae and Plutella xylostella, is substantially heat stable to 55° C., is proteinaceous, acts synergistically with B.thuringiensis cells as an oral pesticide and is substantially resistant to proteolysis by trypsin and proteinase K.

By ‘substantially heat stable to 55° C.’ is meant that the agent retains some pesticidal activity when tested after heating the agent in suspension to 55° C. for 10 minutes, and preferably retains at least 50% of the untreated activity.

By ‘substantially resistant to proteolysis’ is meant that the agent retains some pesticidal activity when exposed to proteases at 30° C. for 2 hours and preferably retains at least 50% of the untreated activity.

By ‘acts synergistically’ is meant that the activity of the combination of components is greater than one might expect from the use of the components individually. For example, when used in conjunction with B. thuringiensis cells as an oral pesticide, the concentration of B. thuringiensis cellular material necessary to give 50% mortality in a P. brassicae when used alone is reduced by at least 80% when it is used in combination the agent at a concentration sufficient to give 25% mortality when the agent is used alone.

It has been found that the activity of the material is retained by 30 kDa cut-off filters but is only partly retained by 100 kDa filters.

Preferably the agent is still further characterised in that the pesticidal activity is lost through treatment at 25° C. with sodium dodecyl sulphate (SDS—0.1% 60 mins) and acetone (50%, 60 mins).

Clearly the characterising properties of the isolated agent described above can be utilised to purify it from, or enrich its concentration in, Xenorhabdus species cells and culture medium supernatants. Methods of purifying proteins from heterogenous mixtures are well known in the art (eg. ammonium sulphate precipitation, proteolysis, ultrafiltration with known molecular weight cut-off filters, ion-exchange chromatography, gel filtration, etc.). The oral pesticidal activity provides a convenient method of assaying the level of agent after each stage, or in each sample of eluent. Such methodology does not require inventive endeavour by those skilled in the art.

The invention further discloses oral pesticidal compositions comprising one or more agents as described above. Such compositions preferably further comprise other pesticidal materials from non-Xenorhabdus species.

These other materials may be chosen such as to have complementary properties to the agents described above, or act synergistically with it.

Preferably the oral pesticidal composition comprises one or more pesticidal agents as described above in combination with B. thuringiensis (or with a toxin derived therefrom, preferably endotoxin).

Recombinant DNA encoding said proteins also forms a further aspect of the invention. The DNA may be incorporated into an expression vector under the influence of suitable control elements such as promoters, enhancers, signal sequences etc. as is understood in the art. These expression vectors form a further aspect of the invention. They may be used to transform a host organism so as to ensure that the organism produces the toxin.

The invention further makes available a host organism comprising a nucleotide sequence coding for a pesticial agent as described above.

Methods of cloning the sequence for a characterised protein into a host organism are well known in the art. For instance the protein may be purified and sequenced: as activity is not required for sequencing, SDS gel electrophoresis followed by blotting of the gel may be used to purify the protein. The protein sequence can be used to generate a nucleotide probe which can itself be used to identify suitable genomic fragments from a Xenorhabdus gene library. These fragments can then be inserted via a suitable vector into a host organism which can express the protein. The use of such general methodology is routine and non-inventive to those skilled in the art. Such techniques may be applied to the production of Xenorhabdus toxins other than those encoded by the sequence of FIG. 2.

It may be desirable to manipulate (eg. mutate) the agent by altering its gene sequence (and hence protein structure) such as to optimise its physical or toxicological properties.

It may also be desirable for the host to be engineered or selected such that it also expresses other proteinaceous pesticidal materials (eg. delta-endotoxin from B. thuringiensis). Equally it may be desirable to generate host organisms which express fusion proteins composed of the active portion of the agent plus these other toxicity enhancing materials.

A host may be selected for the purposes of generating large quantities of pesticidal materials for purification e.g. by using B. thuringiensis transformed with the agent-coding gene. Preferably however the host is a plant, which would thereby gain improved pest-resistance. Suitable plant vectors, eg. the Ti plasmid from Agrobacterium tumefaciens, are well known in the art. Alternatively the host may be selected such as to be directly pathogenic to pests, eg. an insect baculovirus.

The teaching and scope of the present invention embraces all of these host organisms plus the agents, mutated agents or agent-fusion materials which they express.

Thus the invention makes available methods, compositions, agents and organisms having industrially applicable pesticidal activity, being particularly suited to improved crop protection or insect-mediated disease control.

The methods, compositions and agents of the present invention will now be described, by way of illustration only, through reference to the following non-limiting examples and figures. Other embodiments falling within the scope of the invention will occur to those skilled in the art in the light of these.

### FIGURE

FIG. 1 shows the variation with time of the growth of X. nematophilus ATCC 19061 and activity of cells and supernatants against P. brassicae as described in Example 3.

FIG. 2 shows the sequence of a major part of a cloned toxin gene from Xenorhabdus.

FIG. 3 shows a comparison of the restriction maps of cloned toxin genes from two strains of Xenorhabdus (clone 1 above and clone 3 below).

### EXAMPLES

### Example 1

Use of X. nematophilus Cells as an Oral Insecticide

CELL GROWTH: A subculture of X. nematophilus (ATCC 19061, Strain 9965 available from the National Collections of Industrial and Marine Bacteria, Aberdeen, Scotland) was used to inoculate 250 ml Erlenmeyer flasks each containing 50 ml of Luria Broth containing 10 g tryptone, 5 g yeast extract and 5 g NaCl per litre. Cultures were grown in the flasks at 27° C. for 40 hrs on a rotary shaker.

PRODUCTION OF CELL SUSPENSION: Cultures were centrifuged at 5000×g for 10 mins. The supernatants were discarded and the cell pellets washed once and resuspended in an equal volume of phosphate buffered saline (8 g NaCl, 1.44 g Na₂HPO₄ and 0.24 g of KH₂PO₄ per litre) at pH 7.4.

ACTIVITY OF CELL SUSPENSION TO INSECTS: The bioassays were as follows: P. brassicae: The larvae were allowed to feed on an artificial agar-based diet (as described by David and Gardiner (1965) London Nature, 207, 882-883) into which a series of dilutions of cell suspension had been incorporated. The bioassays were performed using a series of 5 doses with a minimum of 25 larvae per dose. Untreated and heat-treated (55° C. for 10 minutes) cells were tested. Mortality was recorded after 2 and 4 days with the temperature maintained at 25° C.

LC50 cells/g diet

| Treatment | 2 days | 4 days |
|---|---|---|
| Untreated | 5.9 × 10⁵ | 9.8 × 10⁴ |
| Treated 55° C. | 7.1 × 10⁵ | 1.4 × 10⁵ |

Aedes aegypti: The larva were exposed to a series of 5 different dilutions of cell suspension in deionised water. The biosassays were performed using 2 doses per dilution of 50 ml cell suspension in 9.5 cm plastic cups with 25 second instar larvae per dose. Untreated and heat-treated (55° C. or 80° C. for 10 minutes) cells were tested. Mortality was recorded after 2 days with the temperature maintained at 25° C.

| | LC50 cells/ml |
|---|---|
| Treatment | 2 days |
| Untreated | 5.1 × 10⁶ |
| Treated 55° C. | 7.4 × 10⁶ |
| Treated 80° C. | >10⁸ |

Culex quinquefaciatus: The larvae were exposed to a single concentration cell suspension containing 4×10⁷ cells/ml. The biosassays were performed using 2 50 ml cell suspensions in 9.5 cm plastic cups with 25 second instar larvae per cup. Untreated and heat-treated (55° C. or 80° C. for 10 minutes) cells were tested. Mortality was recorded after 2 days with the temperature maintained at 25° C.

| | % Mortality |
|---|---|
| Treatment | 2 days |
| Untreated | 100 |
| Treated 55° C. | 100 |
| Treated 80° C. | 0 |

Thus these results clearly show that cells from X. nematophilus are effective as an oral insecticide against a number of insect species (and are particularly potent against P. brassicae). The insecticidal activity is not dependent on cell viability (i.e is largely unaffected by heating to 55° C. which reduces cell viability by >99.99%) but is much reduced by heating to 80° C., which denatures most proteins.

### Example 2

Use of X. nematophilus Supernatant as an Oral Insecticide

CELL GROWTH: Cultures were grown as in Example 1.

PRODUCTION OF SUPERNATANT: Cultures were centrifuged twice at 10000 g for 10 mins. The cell pellets were discarded.

Activity of Supernatant to Insects: The Bioassay Was as Follows:

Activity against neonate P. brassicae and two day old Pieris rapae and Plutella xylostella larvae was measured as for P. brassicae in Example 1, but using a series of untreated dilutions of supernatant in place of of cell supensions and with mortality being recorded after 4 days only.

| | LC50 (μl supernatant/g diet) |
|---|---|
| Insect species | 4 days |
| P. brassicae | 22 |
| P. rapae | 79 |
| P. xylostella | 135 |

In addition, size-reducing activity (62% reduction in 7 days) against Mamestra brassicae was detected in larvae fed on an artificial diet containing X. nematophilus supernatant (results not shown).

Thus these results clearly show that the supernatant from X. nematophilus culture medium is effective as an oral insecticide against a number of insect species, and are particularly potent against P. brassicae.

The heating of supernatants to 55° C. for 10 minutes caused a partial loss of activity while 80° C. caused complete loss of activity. Activity was also completely lost by treatment with SDS (0.1% w/v for 60 mins) and Acetone (50% v/v for 60 mins) but was unaffected by Triton X-100 (0.1% 60 mins), non-diet P40 (0.1% 60 mins), NaCl (1 M for 60 mins) or cold storage at 4° C. or −20° C. for 2 weeks. All of these properties are consistent with a proteinaceous agent.

The general mode of action of X. nematophilus cells and supernatants i.e. reduction in larval size and death within 2 days at high dosages, and other properties, eg. temperature resistence, appear to be similar suggesting a single agent or type of agent may be responsible for the oral insecticide activity activities of both cells and supernatants.

### Example 3

Timescale for Appearance of Ingestable Insecticidal Activity

CELL GROWTH: 1 ml of an overnight culture of X. nematophilus was used to inoculate an Erlenmeyer flask. Cells were then cultured as in Example 1. Growth was estimated by measuring the optical density at 600 nm.

PRODUCTION OF CELL SUSPENSION AND SUPERNATANTS: These were produced as in Examples 1 and 2.

ACTIVITY OF CELLS AND SUPERNATANTS AGAINST P. BRASSICAE:

The cell suspension bioassay was carried out as in Example 1, but using a single dose of suspended cells to 50 μl of broth/g diet and measuring mortality after 2 days. The cell supernatant bioassay was carried out as in Example 2, but using a single dose equivalent to 50 μl supernatant/g diet (i.e. more than twice the LC50) and measuring mortality after 2 days.

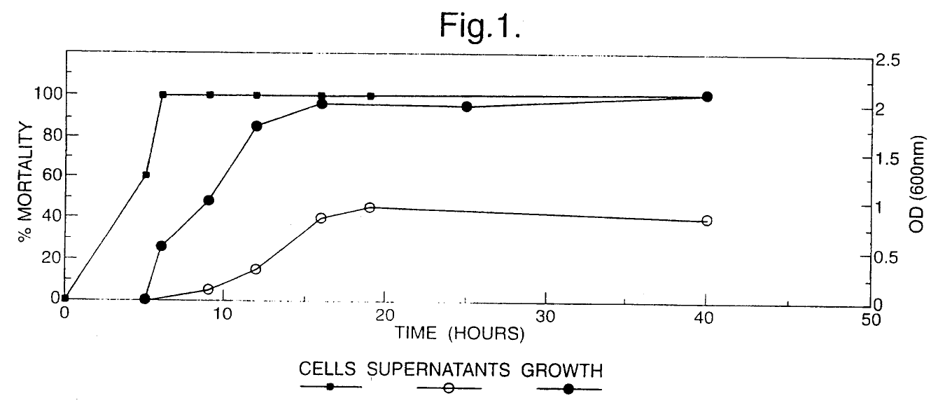

The results are shown in FIG. 1. Thus these results clearly show that cells taken from X. nematophilus culture medium are highly effective as an oral insecticide against P. brassicae after only 5 hours, and supernatants are highly effective after 20 hours. Although some slight cell lysis was observed in the early stages of growth, no significant cell lysis was observed after this point demonstrating that the supernatant activity may be due to an authentic extracellular agent (as opposed to one released only after cell breakdown).

### Example 4

Synergy between X. nematophilus Cells and B. thuringiensis Powder Preparations

CELL GROWTH AND SUSPENSION: X. nematophilus cells were grown and suspended as in Example 1. B. thuringiensis strain HD1 (from Bacillus Genetic Stock Centre, The Ohio State University, Columbus, Ohio 43210, USA) was cultured, harvested and formulated into a powder as described by Dulmage et al.(1970) J. Invertebrate Pathology 15, 15-20.

ACTIVITY OF X. NEMATOPHILUS CELLS AND B. THURINGIENSIS POWDER AGAINST P. BRASSICAE: The bioassays was carried out using X. nematophilus and B. thuringiensis in combination or using B. thuringiensis cell powder alone. Bioassays were carried out as in Example 1 but with various dilutions of B. thuringiensis powder in place of X. nematophilus. For the combination experiment, a constant dose of X. nematophilus cell suspension sufficient to give 25% mortaility was also added to the diet. Mortality was recorded after 2 days.

| | LC50 (μg Bt powder/g diet) |
|---|---|
| Bioassay | 2 days |
| B.t. alone | 1.7 |
| B.t. plus X. nematophilus | 0.09 |

These results clearly demonstrate the synergism between X. nematophilus cells and B. thuringiensis powder when acting as an oral insecticide against P. brassicae.

### Example 5

Synergy Between of X. nematophilus Supernatants and B. thuringiensis Powder

CELL GROWTH AND PRODUCTION OF SUPERNATANTS: X. nematophilus cells were grown and supernatants prepared as in Example 2. B. thuringiensis was grown and treated as in Example 4.

Activity of X. nematophilus Supernatants and Bt Cell Powder Against P. brassicae:

The bioassays were carried out using X. nematophilus supernatants and B. thuringiensis in combination or using B. thuringiensis powder alone. The Bioassay against neonate P. brassicae and two day old Pieris rapae and Plutella xylostella larvae were measured as in Example 2 but with various dilutions of B. thuringiensis in place of X. nematophilus. For the combination experiment, a constant dose of X. nematophilus supernatant sufficient to give 25% mortality was also added to the diet. Mortality was recorded after 4 days.

LC₅₀ (μg Bt powder/g) diet

| Insect species | Bt alone | Bt plus Xn |
|---|---|---|
| P. brassicae | 1.4 | 0.12 |
| P. rapae | 2.5 | 0.26 |
| P. xylostella | 7.2 | 0.63 |

These results clearly demonstrate the synergism between X. nematophilus supernatants and B. thuringiensis powder when acting as an oral insecticide against several insect species. The fact that both X. nematophilus cells and supernatants demonstrate this synergism strongly suggests that a single agent or type of agent is responsible for the demonstrated activities.

### Example 5

Characterisation of Insecticidal Agent from X. nematophilus Supernatant by Proteolysis

CELL GROWTH AND PRODUCTION OF SUPERNATANTS: X. nematophilus cells were grown and supernatants prepared as in Example 2.

PROTEOLYSIS OF SUPERNATANT: Culture supernatant (50 ml) was dialysed against 0.5 M NaCl (3×1 l) for 48 hours at 4° C. The volume of the supernatant in the dialysis tube was reduced five-fold by covering with polyethylene glycol 8000 (Sigma chemicals). Samples were removed and treated with either trypsin (Sigma T8253=10,000 units/mg) or proteinase K (Sigma P0390=10 units/mg) at a concentration of 0.1 mg protease/ml sample for 2 hours at 30° C.

ACTIVITY OF PROTEASE TREATED SUPERNATANT AGAINST P. BRASSICAE: The boassay against neonate P. brassicae larvae was carried out by spreading 25 μl of each ‘treatment’ on the artificial agar-based diet referred to in Example 1 in a 4.5 cm diameter plastic pot. Four pots each containing 10 larvae were used for each treatment. Mortalities were recorded after 1 and 2 days. Controls using water only, trypsin (0.1 mg/ml) and proteinase K (0.1 mg/ml) were also tested in the same way.

% Mortality

| Treatment | 1 day | 2 days |
|---|---|---|
| Untreated supernatant | 60 | 100 |
| Proteinase K treated supernatant | 45 | 100 |
| Trypsin treated supernatant | 40 | 100 |
| All controls (no supernatant) | 0 | 0 |

### Example 6

Entomocidal Activity of Other Xenorhabdus

Using the methodology of Examples 1 and 2, four different xenorhabdus strains were tested against insect pests. The results obtained were as follows:

I) Activity to Pieris brassicae

| Strain deposit | Cells 10⁶/grm diet | Supernatant LC50 |
|---|---|---|
| no/code | % mortality | μl/gram of diet |
| NCIMB 40887 | 100 | 0.09 |
| 0014 | 100 | 0.52 |
| 0015 | 80 | 3.73 |
| NCIMB 40886 | 100 | 0.05 |

It was found that entomocidal activity of cells and supernatant was reduced by more than 99% when all four strains were heated at 80° C. for 10 minutes.

II) Activity to mosquitoes (Aedes aegypti) Bacteria added at the rate of 10⁷ cells/ml of water

| Strain deposit | Cells 10⁶/grm diet |
|---|---|
| no/code | % mortality |
| NCIMB 40887 | 0 |
| 0014 | 40 |
| 0015 | 45 |
| NCIMB 40886 | 95 |

Furthermore, all strains significantly reduced the growth of Heliothis virescens.

### Example 7

Cloning of Toxin Genes from Strains of Xenorhabdus

Total cellular DNA was isolated from NCIMB 40887 and ATCC 19061 using a Quiagen genomic purification DNA kit. Cells were grown in L borth (10 g tryptone, 5 g yeast extract and 5 g NaCl per l) at 28° C. with shaking (150 rpm) to an optical density of 1.5 A₆₀₀. Cultures were harvested by centrifugation at 4000×g and resuspended in 3.5 mls of buffer B1 (50 mM Tris/HCl, 0.05% Tween 20, 0.5% Triton X-100, pH7.0) and incubated for 30 mins at 50° C. DNA was isolated from bacterial lysates using Quiagen 100/G tips as per manufacturers instructions. The resulting purified DNA was stored at −20° C. in TE buffer (10 mM Tris, 1 mM EDTA, pH 8.0).

A representative DNA library was produced using total DNA of NCIMB 40887 and ATTC 19061 partially digested with the restriction enzyme Sau3a. Approximately 20 μg of DNA from each strain was incubated at 37° C. with 0.25 units of the enzyme. At time intervals of 10, 20, 30, 45 and 60 minutes, samples were withdrawn and heated at 65° C. for 15 minutes. To visualise the size of the DNA fragments, the samples were electrophoresed on 0.5% w/v agarose gels.

The DNA samples which contained the highest proportion of 30 to 50 kb fragments were combined and treated with 4 units of shrimp alkaline phosphatase (Boehringer) for 15 minutes at 37° C., followed by heat treatment at 65° C. to inactivate the phosphatase.

The size selected DNA fragments were ligated into the BamH1 site of the cosmid vector SuperCos! (Stratagent) and packaged into the Escherichia coli strain XL Blue 1, using a Gigapack II packaging kit (Stratgene) in accordance with the manufacturers instructions.

To select for cosmid clones with entomocidal activity, individual colonies selected on L agar plates containing 25 μg/ml ampicillin, were grown in L broth (containing 25 μg/ml ampicillin) overnight at 28° C. Broth cultures (50 μl) were individually spread onto the surface of insect diet contained in 4.5 cm diameter pots, as described in Example 5. To each container 10 neonate P. brassicae larvae were added. Larvae were examined after 24, 72 and 96 hours recording mortality and size of surviving larvae. A total of 220 clones of NCIMB 40887 were tested, of which two were found to cause reduction in larval growth and death within 72 hours. Of 370 clones from ATTC 19061, one was found to cause larval death within 72 hours.

### Example 8

Activity of Cloned Toxin Genes to Pieris brassicae

The three active clones from Example 7 were grown in L broth, containing 25 μg/ml ampicillin, for 24 hours at 28° C., on a rotary shaker at 150 rpm. The activity of the toxin clones to neonate larvae were performed by incorporation of whole broth cultures into insect diet, as described in Example 1.

| Clone No | Strain | LC50 (μl broth/g insect diet) |
|---|---|---|
| 1 | NCIMB 40887 | 13.03 |
| 2 | NCIMB 40887 | 16.7 |
| 3 | ATTC 19061 | 108.7 |
| Control* | | No effect at 100 μl/g |

*XL1 Blue E. coli broth

When E. coli toxin clones were heated at 80° C. for 10 minutes and added to the diet at a rate of 100 μl/g, no activity to larvae was detected. Highlighting the heat sensitivity of the toxins.

### Example 9

Sequencing of the Cloned Toxin from NCIMB 40887

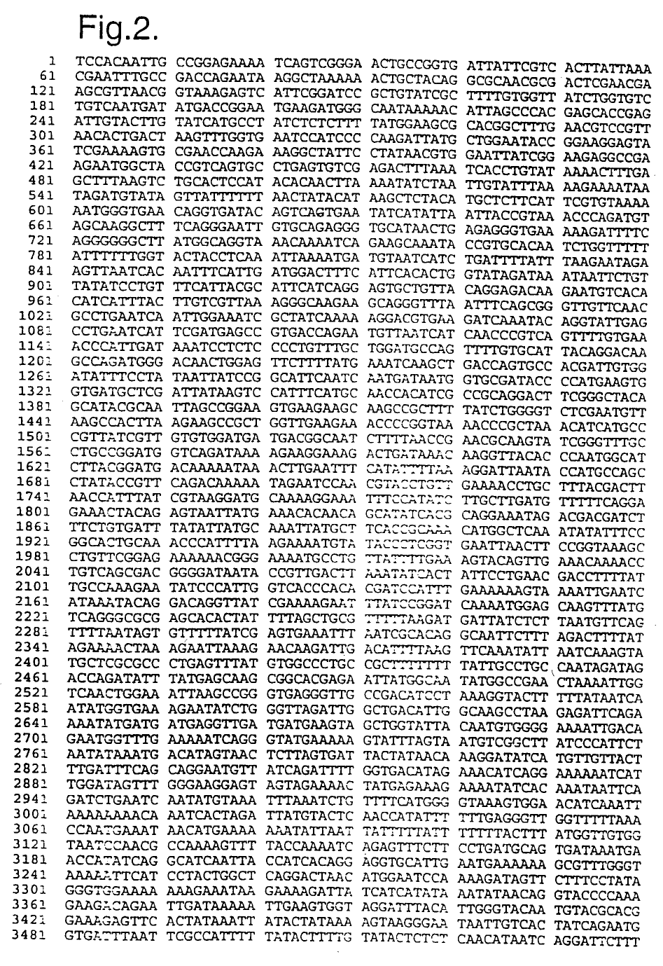

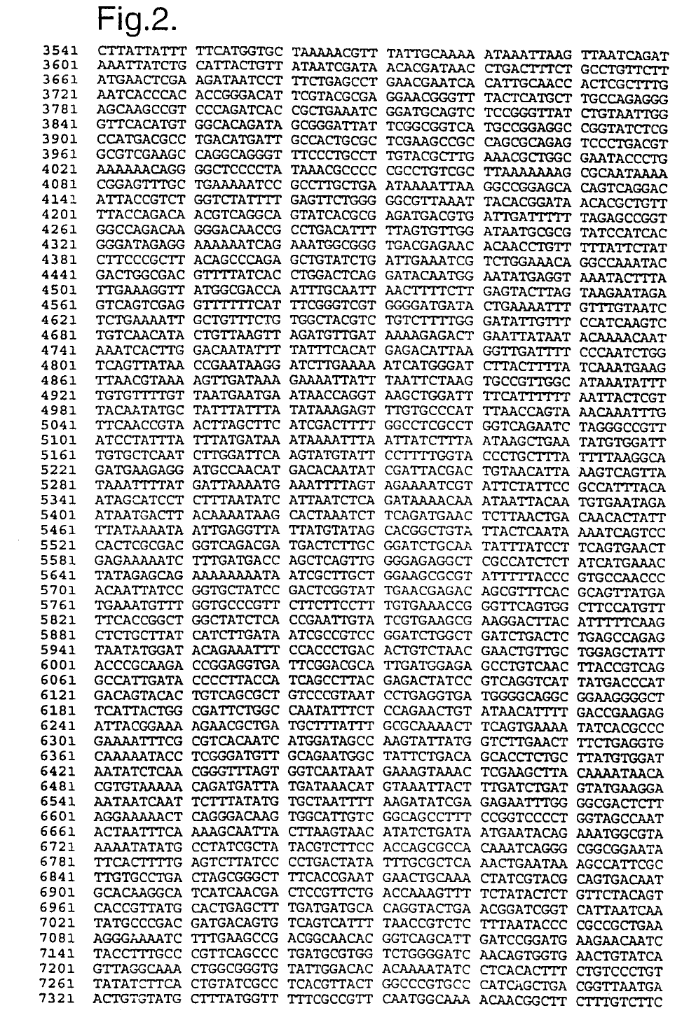

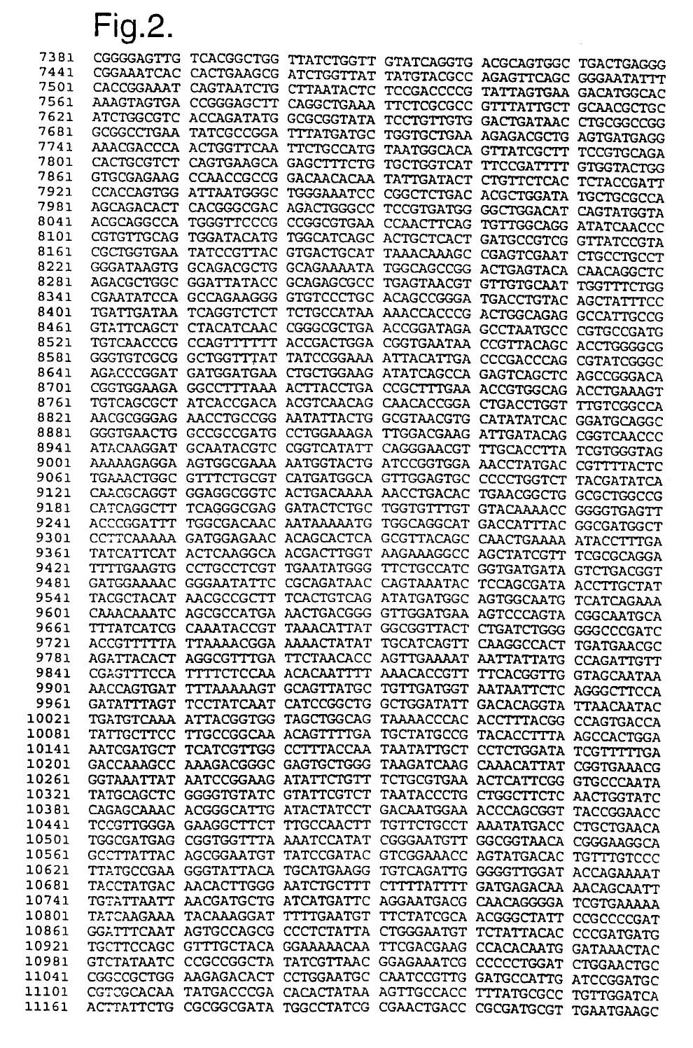

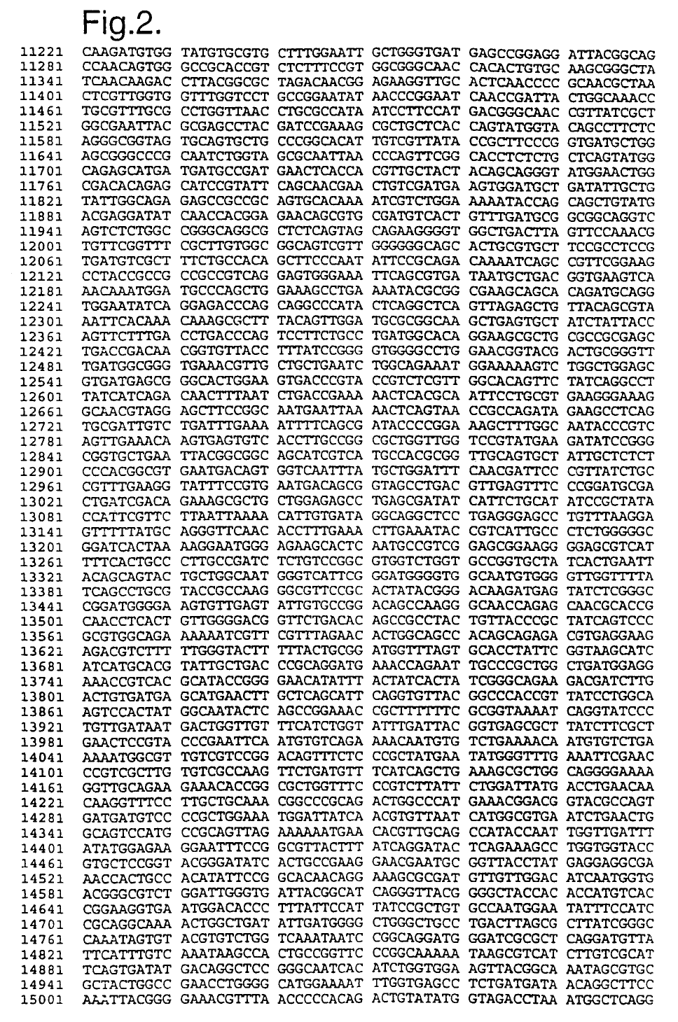

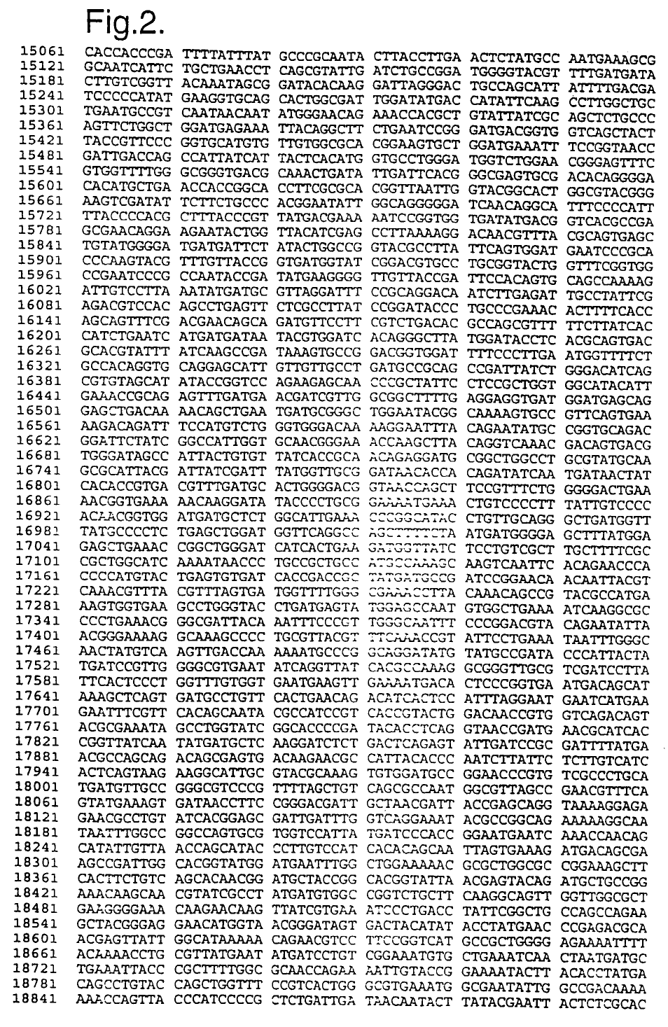

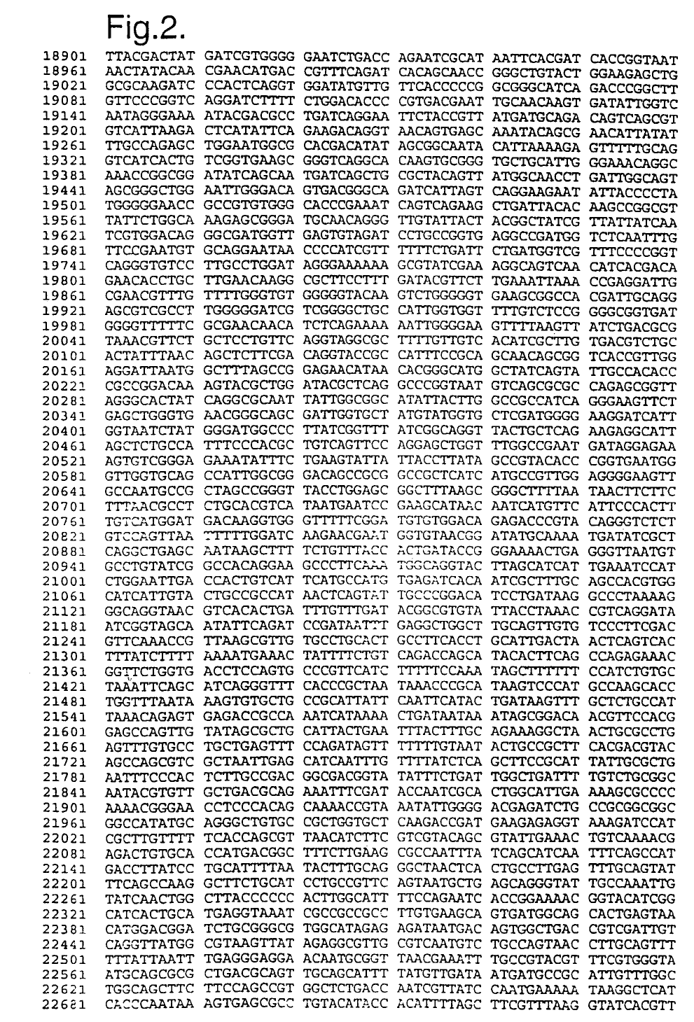

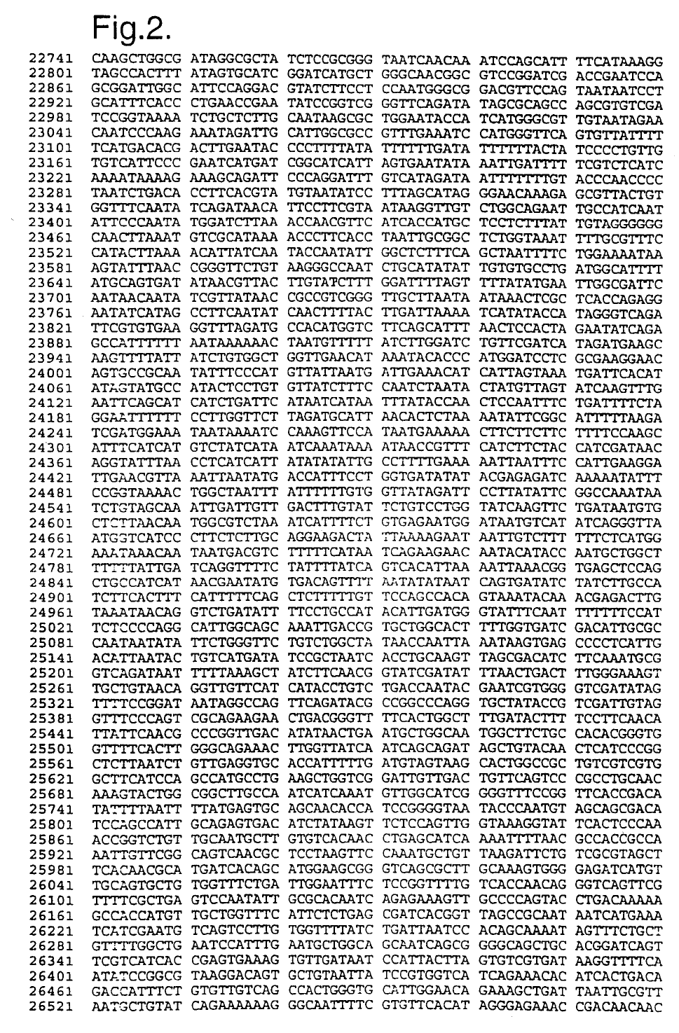

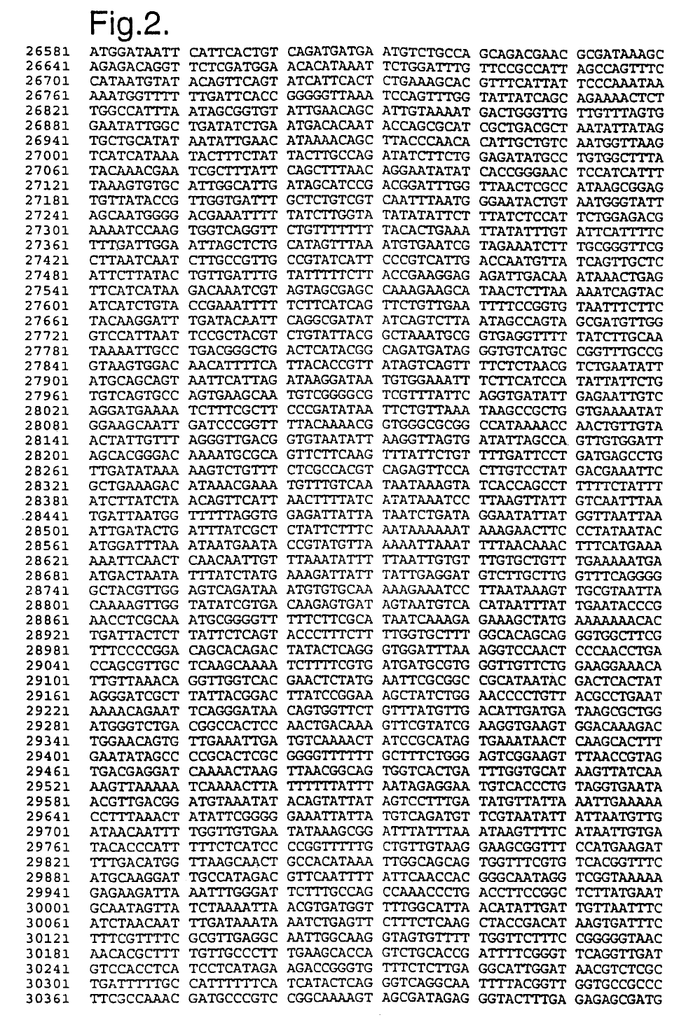

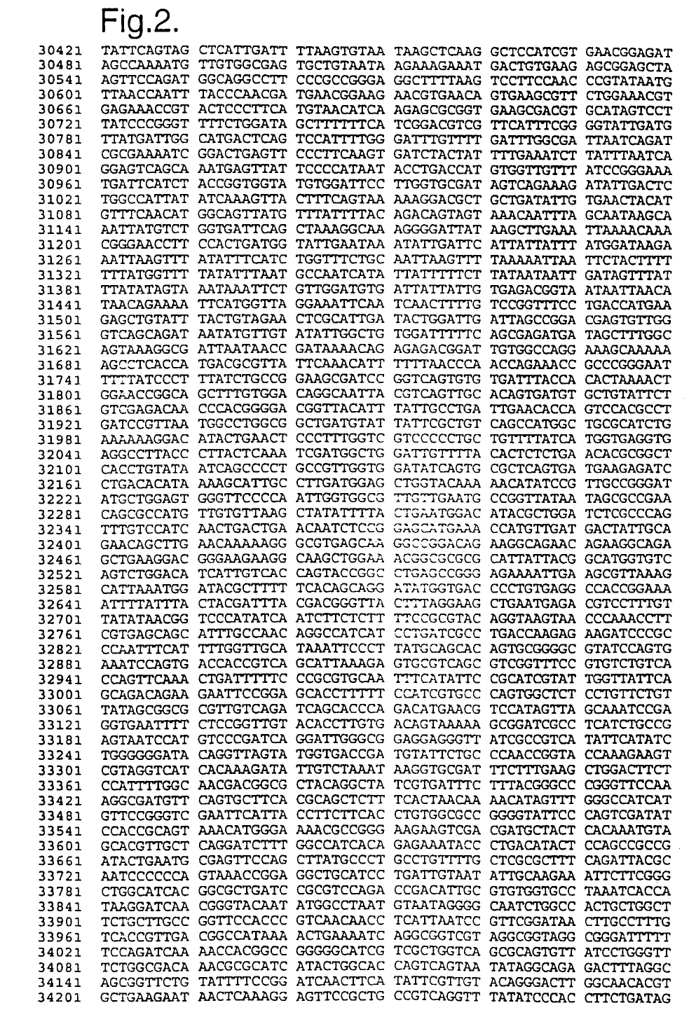

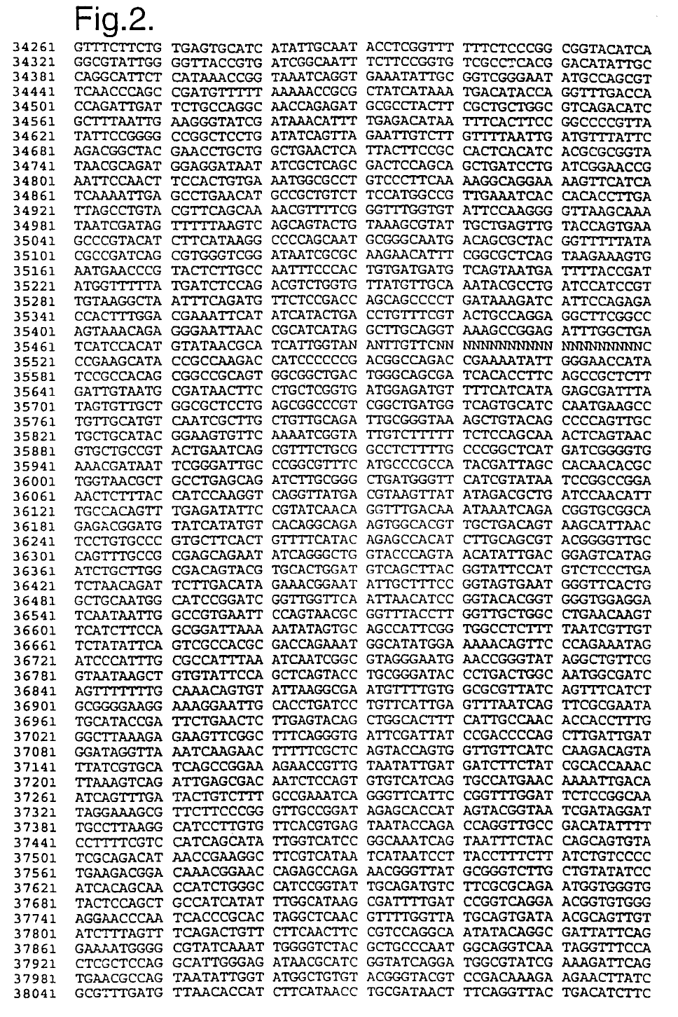

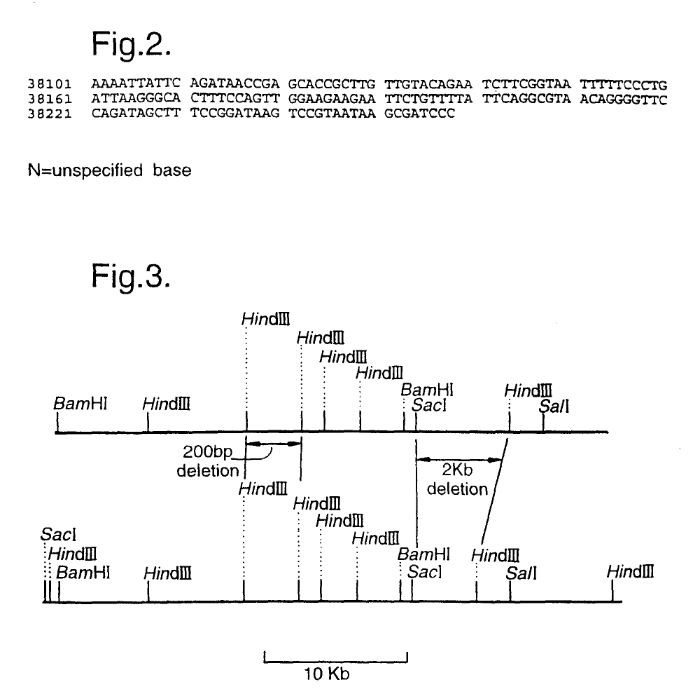

Cosmid DNA of the entomocidal clone 1 above from NCIMB 40887 was purified using the Wizard Plus SV DNA system (Promega) in accordance with the manufacturers instructions. A partial map of the cloned fragment was obtained using a range of restriction enzymes EcoR1, BamH1, HindIII, Sal1 and Sac1 as shown in FIG. 3. DNA sequencing was intiatiated from pUC18 and pUC19 based sub-clones of the cosmid, using the enzymes EcoR1, BamH1, HindIII, EcoRV and PvuII. Sequence gaps were filled using a primer walking approach on purified cosmid DNA. Sequence reactions were performed using the ABI PRISM™ Dye Terminator Cycle Sequencing Ready Reaction Kit with AmmpliTaq DNA polymerase FS according to the manufacturers instructions. The samples were analysed on an ABI automated sequencer according to the manufacturers instructions. The major part of the DNA sequence for the cloned toxin fragment is shown in FIG. 2.

### Example 10

Restriction Map of Cloned Toxin from Clone 3

Cosmid DNA of the entomocidal clone 3 above was purified as described in Example 9. A restriction map of the cloned fragment was obtained using the restriction enzymes BamH1, HindIII, Sal1 and Sac1 and this is shown in FIG. 3. When compared with the map from clone 1 (FIG. 3) it is clear that over the regions which overlap, the restriction maps are very similar. The only detectable difference between the two clones was a reduction in size of two HindIII fragments in clone 3, corresponding to the 11.4 kb and 7.2 kb HindIII fragments in clone 1 by approximately 2 Kb and 200 bp respectively. These results indicate the overall relatedness of the DNA region coding for toxicity in the two bacterial strains.

### Example 11

Southern Blot Hybridisation Experiments

A 10.3 kb BamH1-Sal1 fragment of the DNA from clone 1 was used as a probe to hybidise to total HindIII digested DNA of the Xenorhabdus strains ATCC 19061, NCIMB 40886 and NCIMB 40887. Hybridisation was performed with 20 ng/ml of DIG labelled DNA probe at 65° C. for 18 hours. Filters were washed prior to immunological detection twice for 5 minutes with 2×SSC (0.3M NaCl, 30 mM sodium citrate, pH 7.0)/0.1% (w/v) sodium dodecyl sulphate at room temperature, and twice for 15 minutes with 0.1×SSC (15 mM NaClm 1.5 mM sodium citrate, pH 7.0) plus 0.1% sodium dodecyl sulphate at 65° C. The probe was labelled and experiments performed in accordance with manufacturers instructions, using a non-radioactive DIG DNA labelling and detection kit (Boehringer). The probe hybridised to a HindIII fragment of approximately 8 kb in all three strains as well as an 11.4 kb fragment in NCIMB 40887 and an approximate 9 kb fragment in both NCIMB 40886 and ATCC 19061. These results show that strains NCIMB 40886 and ATCC 19061 contain DNA with close homology to the toxin gene of clone 1 above, confirming the similarity between the toxins produced by the three strains.

1. An insecticidal composition adapted for oral administration to an insect comprising a pesticidal material obtainable from a Xenorhabdus species, or a pesticidal fragment thereof, or a pesticidal variant or derivative of either of these.

2. A composition according to claim 1 wherein the said pesticidal material comprises material encoded by the nucleotide sequence of FIG. 2 or variant or fragment thereof, or a sequence which hybridises with said sequence.

3. A composition according to claim 1 or claim 2 which comprises cells of Xenorhabdus.

4. A composition as claimed in any one of the preceding claims which comprises supernatant taken from cultures of cells of Xenorhabdus species.

5. A composition according to any one of the preceding claims wherein the Xenorhabdus species is Xenorhabdus nematophilus.

6. A composition according to any one of claims 1 to 4 wherein the Xenorhabdus species is ATCC 19061, NCIMB 40886 or NCIMB 40887.

7. A composition as claimed in any one of the preceding claims which comprises a further pesticidal material not obtainable from Xenorhabdus.

8. A composition according to claim 7 wherein the said further pesticidal material comprises a material obtainable from B. thuringiensis.

9. A composition according to claim 8 which further comprises cells of B. thuringiensis.

10. A composition according to claim 8 wherein the pesticidal materials obtainable from B. thuringiensis comprises the delta endotoxin.

11. A composition according to any one of the preceding claims which further comprises an agriculturally acceptable carrier.

12. A composition according to claim 10 wherein the carrier comprises items of insect diet.

13. A method for killing or controlling insect pests, which method comprises administering to a pest or the environment thereof a composition according to any one of the preceding claims.

14. A method as claimed in claim 12 wherein the pests are insects from the order Lepidoptera or Diptera.

15. A microorganism comprising Xenorhabdus strain NCIMB 40886.

16. A microorganism comprising Xenorhabdus strain NCIMB 40887.

17. A pesticidal agent which comprises a a toxin comprising a protein which is encoded by DNA which includes SEQ ID No. 1 or a variant or fragment thereof.

18. An isolated pesticidal agent characterised in that it is obtainable from cultures of X. nematophilus or mutants thereof, has oral pesticidal activity against Pieris brassicae, Pieris rapae and Plutella xylostella, is substantially heat stable to 55° C., is proteinaceous, acts synergistically with B. thuringiensis cells as an oral pesticide, and is substantially resistant to proteolysis by trypsin and proteinase K.

19. An isolated pesticidal agent as claimed in claim 18 further characterised in that the pesticidal activity is substantially destroyed by treatment with sodium dodecyl sulphate or acetone or heating to 80° C.

20. An isolated pesticidal agent as claimed in claim 18 or claim 19 further characterised in that the agent is an extracellular protein.

21. A recombinant DNA which encodes a pesticidal agent according to any one of claims 17 to 20.

22. A recombinant DNA of claim 21 which comprises the sequence of FIG. 2 or a variant or fragment thereof.

23. A recombinant DNA which comprises or hybridises under stringent conditions with all or part of the sequence of FIG. 2, and which encodes a pesticidal material.

24. An expression vector comprising a recombinant DNA according to any one of claims 21 to 23.

25. A host organism which has been transformed with an expression vector according to claim 24.

26. A host organism as claimed in claim 25 which has been engineered or selected such that it also expresses other pesticidal proteinaceous toxicity enhancing materials

27. A host organism comprising a nucleotide sequence coding for a fusion protein comprising a pesticidally active portion of an agent as claimed in any one of claims 17 to 20 in combination with other pesticidal proteinaceous toxicity enhancing materials.

28. A host organism as claimed in claim 27 wherein the pesticidal toxicity enhancing materials comprise delta-endotoxin from B. thuringiensis.

29. A host organism as claimed in any one of claims 25 to 28 wherein the host is a plant.

30. A host organism as claimed in any one of claims 25 to 28 wherein the host is a virus pathogenic to insects.

31. A fusion protein as expressed by a host as claimed in claim 27.

32. An pesticidal composition comprising one or more agents as claimed in any one of claims 17 to 20.
